(19) United States
(12) Patent Application Publication
(10) Pub. No.: US 2014/0024909 A1
(43) Pub. Date: Jan. 23, 2014

(54) MRI-GUIDED CATHETERS

(71) Applicants: Kamal Vij, Chandler, AZ (US); Parag V. Karmarkar, Columbia, MD (US); Rajesh Pandey, Irvine, CA (US); Peter Piferi, Orange, CA (US)

(72) Inventors: Kamal Vij, Chandler, AZ (US); Parag V. Karmarkar, Columbia, MD (US); Rajesh Pandey, Irvine, CA (US); Peter Piferi, Orange, CA (US)

(73) Assignee: MRI INTERVENTIONS, INC., Memphis, TN (US)

(21) Appl. No.: 13/984,437

(22) PCT Filed: Feb. 24, 2012
(86) PCT No.: PCT/US2012/026468

§ 371 Date: Oct. 8, 2013

Related U.S. Application Data
(60) Provisional application No. 61/446,329, filed on Feb. 24, 2011.

(51) Int. Cl. A61M 25/01; A61B 5/00; A61B 18/18; A61M 5/00; A61B 5/04; A61B 18/14

(52) U.S. Cl. CPC A61M 25/0127; A61B 5/04; A61B 18/1492; A61B 18/18; A61M 5/00; A61B 5/4836
USPC 600/373; 600/411

(57) ABSTRACT

An MRI-compatible catheter reduces localized heating due to MR scanner-induced currents and includes a shaft having opposite distal and proximal end portions. One or more RF tracking coils are positioned adjacent the distal end portion and each includes a conductive lead that electrically connects the RF tracking coil to an MRI scanner. Each lead includes a series of back and forth segments along its length. The catheter includes one or more sensing electrodes at the shaft distal end portion, each electrically connected to a resistor having high impedance. A sheath surrounds a portion of the elongated shaft and includes at least one RF shield coaxially disposed therewithin. Each RF shield includes elongated inner and outer conductors and an elongated dielectric layer of MRI compatible material sandwiched between the inner and outer conductors. The inner and outer conductors are electrically connected at one end and electrically isolated at an opposite end.

### RELATED APPLICATION

This application claims the benefit of and priority to U.S. Provisional Patent Application No. 61/446,329 filed Feb. 24, 2011, which is a continuation-in-part of U.S. patent application Ser. No. 12/816,803 filed Jun. 16, 2010, the disclosures of which are incorporated herein by reference as if set forth in their entireties.

### FIELD OF THE INVENTION

The present invention relates to MRI-guided systems and may be particularly suitable for MRI-guided cardiac systems such as EP systems for treating arrhythmias.

### BACKGROUND

Heart rhythm disorders (arrhythmias) occur when there is a malfunction in the electrical impulses to the heart that coordinate how the heart beats. During arrhythmia, a heart may beat too fast, too slowly or irregularly. Catheter ablation is a widely used therapy for treating arrhythmias and involves threading a catheter through blood vessels of a patient and into the heart. In some embodiments, radio frequency (RF) energy may be applied through the catheter tip to destroy abnormal heart tissue causing the arrhythmia. In other embodiments a catheter tip may be configured to cryogenically ablate heart tissue.

Guiding the placement of a catheter during ablation therapy within the heart is important. Conventional catheter ablation procedures are conducted using X-ray and/or ultrasound imaging technology to facilitate catheter guidance and ablation of heart tissue. Conventional Cardiac EP (ElectroPhysiology) Systems are X-ray based systems which use electroanatomical maps. Electroanatomical maps are virtual representations of the heart showing sensed electrical activity. Examples of such systems include the Carto® electroanatomic mapping system from Biosense Webster, Inc., Diamond Bar, Calif., and the EnSite NavX® system from Endocardial Solutions Inc., St. Paul, Minn.

Magnetic resonance imaging (MRI) has several distinct advantages over X-ray imaging technology, such as excellent soft-tissue contrast, the ability to define any tomographic plane, and the absence of ionizing radiation exposure. In addition, MRI offers several specific advantages that make it especially well suited for guiding various devices used in diagnostic and therapeutic procedures including: 1) real-time interactive imaging, 2) direct visualization of critical anatomic landmarks, 3) direct high resolution imaging, 4) visualization of a device-tissue interface, 5) the ability to actively track device position in three-dimensional space, and 6) elimination of radiation exposure.

Induced RF currents (referred to as RF coupling) on coaxial cables, electrical leads, guide wires, and other elongated devices utilized in MRI environments can be problematic. Such RF coupling may cause significant image artifacts, and may induce undesired heating and cause local tissue damage. To reduce the risk of tissue damage, it is desirable to reduce or prevent patient contact with cables and other conductive devices in an MRI environment. Such contact, however, may be unavoidable in some cases. For devices that are inserted inside the body, such as endorectal, esophageal, and intravascular devices, the risk of tissue damage may increase.

### SUMMARY

It should be appreciated that this Summary is provided to introduce a selection of concepts in a simplified form, the concepts being further described below in the Detailed Description. This Summary is not intended to identify key features or essential features of this disclosure, nor is it intended to limit the scope of the invention.

According to some embodiments of the present invention, an MRI-compatible catheter that reduces localized heating due to MR scanner-induced currents includes an elongated flexible shaft having a distal end portion and an opposite proximal end portion. A handle is attached to the proximal end portion and includes an electrical connector interface configured to be in electrical communication with an MRI scanner. One or more RF tracking coils are positioned adjacent the distal end portion of the shaft. Each RF tracking coil includes a conductive lead, such as a coaxial cable, that extends between the RF tracking coil and the electrical connector interface and electrically connects the RF tracking coil to an MRI scanner. In some embodiments of the present invention, the conductive lead has a length sufficient to define an odd harmonic/multiple of a quarter wavelength of the operational frequency of the MRI Scanner, and/or includes a series of pre-formed back and forth segments along its length. In some embodiments of the present invention, the conductive lead is a coaxial cable that includes a self-resonant cable trap, such as, for example, a 60-turn inductor.

In some embodiments of the present invention, the catheter includes one or more sensing electrodes at the shaft distal end portion. One or more of the sensing electrodes is electrically connected to a high impedance resistor, for example, a resistor having an impedance of, for example, at least about 5,000 ohms.

In some embodiments of the present invention, the catheter includes a tuning circuit that is configured to stabilize tracking signals generated by one or more RF tracking coils. The tuning circuit may be located within the handle of the catheter.

In some embodiments of the present invention, a sheath surrounds at least a portion of the elongated shaft and includes at least one RF shield coaxially disposed therewithin. Each RF shield includes elongated inner and outer tubular conductors. The inner and outer conductors each have respective opposite first and second end portions. An elongated tubular dielectric layer of MRI compatible material is sandwiched between the inner and outer conductors and surrounds the inner conductor. Only the respective first end portions of the inner and outer conductors are electrically connected. The second end portions are electrically isolated from each other. In some embodiments, the inner and outer conductors comprise conductive foil, conductive braid, or a film with a conductive surface. A plurality of RF shields may be disposed within the sheath in end-to-end spaced-apart relationship.

In some embodiments of the present invention, at least one RF shield coaxially disposed within the flexible shaft of the catheter. Each RF shield includes elongated inner and outer tubular conductors. The inner and outer conductors each have respective opposite first and second end portions. An elongated tubular dielectric layer of MRI compatible material is sandwiched between the inner and outer conductors and surrounds the inner conductor. Only the respective first end portions of the inner and outer conductors are electrically connected. The second end portions are electrically isolated from each other. In some embodiments, the inner and outer conductors comprise conductive foil, conductive braid, or a film with a conductive surface. A plurality of RF shields may be disposed within the flexible shaft of the catheter in end-to-end spaced-apart relationship.

According to some embodiments of the present invention, the catheter is an ablation catheter with an ablation tip at the shaft distal end portion. An RF conductor extends longitudinally within the shaft from the ablation tip to the electrical connector interface at the handle and connects the ablation tip to an RF generator. The RF conductor includes a series of pre-formed back and forth segments along its length.

It is noted that aspects of the invention described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail below.

### BRIEF DESCRIPTION OF THE DRAWINGS

The accompanying drawings, which form a part of the specification, illustrate some exemplary embodiments. The drawings and description together serve to fully, explain the exemplary embodiments.

FIG. 1 is a schematic illustration of an MRI-guided system configured to show a device tissue interface using near RT MRI data according to some embodiments of the present invention.

FIG. 2 is a schematic illustration of an intrabody device with a tracking coil electrically connected to an MRI Scanner channel according to embodiments of the present invention.

FIG. 3 is a schematic illustration of an MRI system with a workstation and display according to some embodiments of the invention.

FIG. 4 is a circuit diagram of an exemplary tracking coil tuning circuit according to some embodiments of the present invention.

FIG. 5 is a perspective view of an exemplary ablation catheter, according to some embodiments of the present invention.

FIG. 6 is a perspective view of the handle at the proximal end of the ablation catheter of FIG. 5, according to some embodiments of the present invention, and with a cover removed.

FIG. 7 is a schematic illustration of an exemplary ablation catheter, according to some embodiments of the present invention.

FIG. 8 is a schematic illustration of a self-resonant cable trap that may be utilized by the ablation catheter of FIG. 7, according to some embodiments of the present invention.

FIG. 9 is a cross-sectional view of a sheath with an integrated RF shield disposed therewithin for use with the ablation catheter of FIG. 7, according to some embodiments of the present invention.

FIG. 10 is a partial side view of the distal end of an ablation catheter with a portion in a sheath, such as that shown in FIG. 9.

FIG. 11A is a greatly enlarged perspective view of an RF shield disposed within a sheath, according to some embodiments of the present invention.

FIGS. 11B and 11C are respective opposite end views of the RF shield of FIG. 11A.

FIG. 12 is a partial side view of a sheath including multiple RF shields in end-to-end spaced-apart relationship, according to some embodiments of the present invention.

FIGS. 13-14 are graphs illustrating RF safety performance of a billabong assembly, according to some embodiments of the present invention.

FIG. 15 is a graph illustrating broad spectrum, high attenuation of a billabong assembly, according to some embodiments of the present invention.

FIG. 16 is a schematic illustration of a single layer billabong assembly, according to some embodiments of the present invention.

### DETAILED DESCRIPTION

The present invention will now be described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. It will be appreciated that although discussed with respect to a certain embodiment, features or operation of one embodiment can apply to others.

In the drawings, the thickness of lines, layers, features, components and/or regions may be exaggerated for clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known /functions or constructions may not be described in detail for brevity and/or clarity.

It will be understood that when a feature, such as a layer, region or substrate, is referred to as being “on” another feature or element, it can be directly on the other element or intervening elements may also be present. In contrast, when an element is referred to as being “directly on” another feature or element, there are no intervening elements present. It will also be understood that, when a feature or element is referred to as being “connected” or “coupled” to another feature or element, it can be directly connected to the other element or intervening elements may be present. In contrast, when a feature or element is referred to as being “directly connected” or “directly coupled” to another element, there are no intervening elements present. Although described or shown with respect to one embodiment, the features so described or shown can apply to other embodiments.

The terms “MRI Scanner” and “MR Scanner” are used interchangeably to refer to a Magnetic Resonance Imaging system and includes the magnet, the operating components, e.g., RF amplifier, gradient amplifiers and operational circuitry including, for example, processors (the latter of which may be held in a control cabinet) that direct the pulse sequences, select the scan planes and obtain MR data. Embodiments of the present invention can be utilized with any MRI Scanner including, but not limited to, GE Healthcare: Signa 1.5T (Tesla)/3.0T; Philips Medical Systems: Achieva 1.5T/3.0T; Integra 1.5T; Siemens: MAGNETOM Avanto; MAGNETOM Espree; MAGNETOM Symphony; MAGNETOM Trio; and MAGNETOM Verio.

The term “RF safe” means that the catheter and any (conductive) lead associated therewith is configured to operate safely when exposed to RF signals, particularly RF signals associated with MRI systems, without inducing unplanned current that inadvertently unduly heats local tissue or interferes with the planned therapy.

The term “MRI visible” means that a device is visible, directly or indirectly, in an MRI image. The visibility may be indicated by the increased SNR of the MRI signal proximate the device. The device can act as an MRI receive antenna to collect signal from local tissue and/or the device actually generates MRI signal itself, such as via suitable medical grade hydro-based coatings, fluid (e.g., aqueous fluid) filled channels or lumens.

The term “MRI compatible” means that the so-called component(s) is safe for use in an MRI environment and as such is typically made of a non-ferromagnetic MRI compatible material(s) suitable to reside and/or operate in a high magnetic field environment and produce no MR artifact. MRI compatible devices, according to embodiments of the present invention, may also be bio-compatible so as to be suitable for insertion within the body of a patient

The term “high-magnetic field” refers to field strengths above about 0.5T, typically above 1.0T, and more typically between about 1.5T and 10T. Embodiments of the present invention may be particularly suitable for 1.5T and/or 3.0T systems.

The term “intrabody device” is used broadly to refer to any diagnostic or therapeutic medical device including, for example, catheters, needles (e.g., injection, suture, and biopsy), forceps (miniature), knives or other cutting members, ablation or stimulation probes, injection or other fluid delivery cannulas, mapping or optical probes or catheters, sheaths, guidewires, fiberscopes, dilators, scissors, implant material delivery cannulas or barrels, and the like, typically having a size that is between about 5 French to about 12 French, but other sizes may be appropriate.

The term “tracking member”, as used herein, includes all types of components that are visible in an MRI image including miniature RF tracking coils, passive markers, and receive antennas. In some embodiments of the present invention, at least one miniature RF tracking coil on a catheter can be connected to a channel of an MRI Scanner. The MR Scanner can be configured to operate to interleave the data acquisition of the tracking coils with the image data acquisition. The tracking data can be acquired in a “tracking sequence block” which takes about 10 msec (or less). In some embodiments, the tracking sequence block can be executed between each acquisition of image data (the “imaging sequence block”). So the tracking coil coordinates can be updated immediately before each image acquisition and at the same rate. The tracking sequence can give the coordinates of all tracking coils simultaneously. So, typically, the number of coils used to track a device has substantially no impact on the time required to track them.

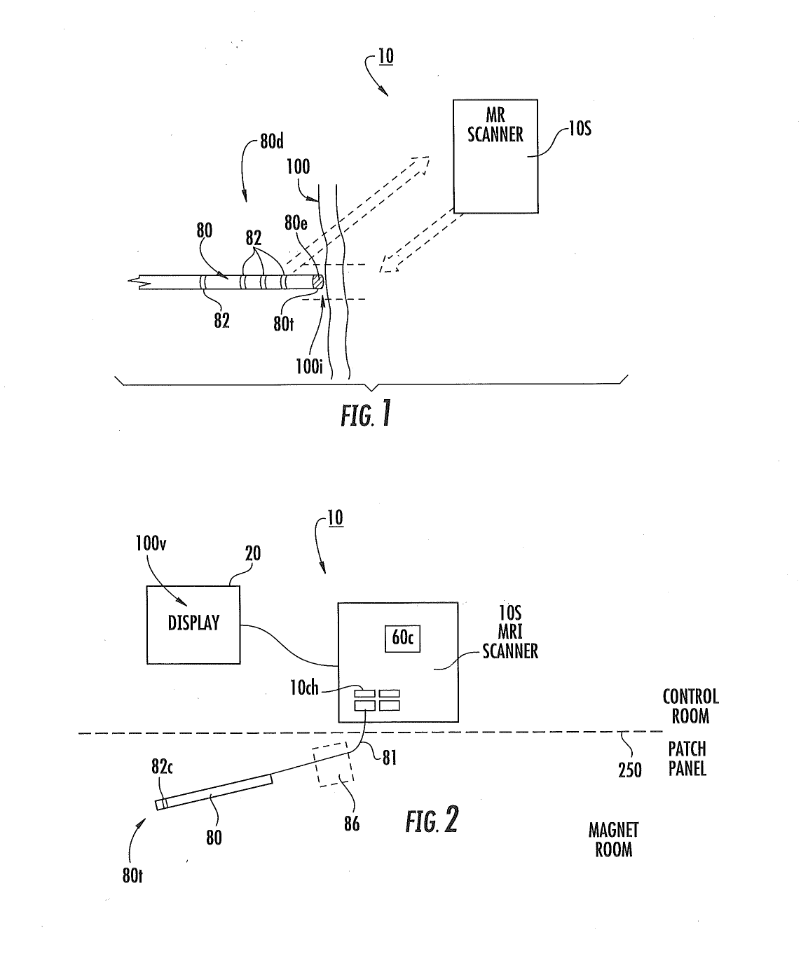

FIG. 1 illustrates an MRI interventional system 10 with a scanner 10S and a flexible intrabody medical device 80 (e.g., an ablation catheter, mapping catheter, etc.) proximate target tissue 100 of a body of a patient at a device-tissue interface 100i. The system 10 can be configured to electronically track the 3-D location of the device 80 in the body of a patient and identify and/or “know” the location of the tip portion 80t of the device 80 (e.g., the ablation tip) in a coordinate system associated with the 3-D imaging space. As shown in FIG. 1, the device 80 can include a plurality of spaced apart tracking members 82 on a distal end portion thereof. In a particular embodiment, the device 80 can be an ablation catheter and the tip 80t can include an ablation electrode 80e (typically at least one at a distal end portion of the device). Where used, the electrode 80e can be both a sensing and ablation electrode or merely an ablation electrode. In some embodiments, the ablation electrode 80e can be formed from copper or copper alloy (e.g., copper alloy 101, available from McMaster-Carr, Santa Fe Springs, Calif.).

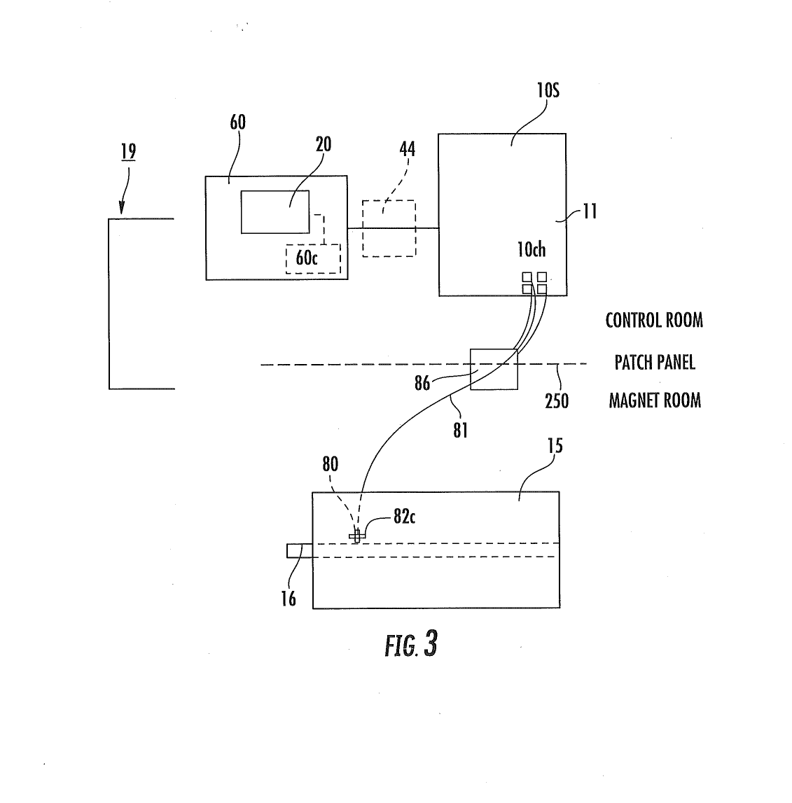

The tracking members 82 can comprise miniature tracking coils, passive markers and/or an antenna. In a preferred embodiment, the tracking members 82 include at least one miniature tracking coil 82c that is connected to a channel 10ch of an MRI Scanner 10S (FIG. 2). The MR Scanner 10S can be configured to operate to interleave the data acquisition of the tracking coils 82c with the image data acquisition. The tracking data is typically acquired in a “tracking sequence block” which takes about 10 msec (or less). In some embodiments, the tracking sequence block can be executed between each acquisition of image data (the latter can be referred to as an “imaging sequence block”). So the tracking coil coordinates can be updated immediately before each image acquisition and at the same rate. As noted above, the tracking sequence can give the coordinates of all tracking coils simultaneously.

The system 10 and/or circuit 60c (FIGS. 2-3) can calculate the position of the tip 80t of the device 80 in the patient body as well as the shape and orientation of the flexible device 80 based on a priori information on the dimensions and behavior of the device 80 (e.g., for a steerable device, the amount of curvature expected when a certain pull wire extension or retraction exists, distance to tip from different coils 82 and the like). Using the known information of the device 80 and because the tracking signals are spatially associated with the same X, Y, Z coordinate system as the MR image data, the circuit 60c can rapidly generate visualizations showing a physical representation of the location of a distal end portion of the device 80 with near real time (RT) MR images of the patient anatomy.

In some embodiments, the tracking signal data is obtained and the associated spatial coordinates are determined while a circuit 60c in the MRI Scanner 10S (FIG. 2) and/or in communication with the Scanner 10S (FIG. 3) obtains MR image data. The reverse operation can also be used. The circuit 60c can then rapidly render the resultant visualization(s) in a display, such as display 20 (FIGS. 2, 3) with the flexible device(s) 80 shown with a physical representation based on spatial coordinates of the devices in the 3-D imaging space identified using the associated tracking coil data and the near RT MR image(s).

The circuit 60c can be totally integrated into the MR Scanner 10S (e.g., control cabinet), partially integrated into the MR Scanner 10S or be separate from the MR Scanner 10S but communicate therewith. If not totally integrated into the MR Scanner 10S, the circuit 60c may reside partially or totally in a workstation 60 and/or in remote or other local processor(s) and/or ASIC (application-specific integrated circuit). FIG. 3 illustrates that a clinician workstation 60 can communicate with the MR Scanner 10S via an interface 44. Similarly, the device 80 in the magnet room can connect to the MR Scanner 10S via an interface box 86 which may optionally be integrated into the patch panel 250.

As shown in FIGS. 2 and 3, for example, the system 10 can include at least one (interactive) display 20 in communication with the circuit 60c and/or the Scanner 10S. The display 20 can be configured to display the interactive visualizations. The visualizations can be dynamic showing the movement of the device 80 relative to the intrabody anatomical structure shown by the displayed near-real time MRI image.

FIG. 2 illustrates that the device 80 can include at least one conductor 81, such as a coaxial cable that connects a respective tracking coil 82c to a channel 10ch of the MR Scanner 10S. The MR Scanner 10S can include at least 16 separate channels, and typically more channels but may operate with less as well. Each device 80 can include between about 1-10 tracking coils, typically between about 1-4. The tracking coils 82c on a particular device 80 can be arranged with different numbers of turns, different dimensional spacing between adjacent coils 82c (where more than one coil is used) and/or other configurations. The circuit 60c can be configured to generate the device renderings based on tracking coil locations/positions relative to one another on a known device with a known shape and/or geometry or predictable or known changeable (deflectable) shape or form (e.g., deflectable end portion). The circuit 60c can identify or calculate the actual shape and orientation of the device for the renderings based on data from a CAD (computer aided design) model of the physical device. The circuit 60c can include data regarding known or predictable shape behavior based on forces applied to the device 80 by the patient body or by internal or external components and/or based on the positions of the different tracking coils 82c in 3-D image space and known relative (dimensional) spacings.

As shown in FIG. 3, the display 20 can be provided in or associated with a clinician workstation 60 in communication with an MRI Scanner 10S. Other displays may be provided. The MRI Scanner 10S typically includes a magnet 15 in a shielded room and a control cabinet 11 (and other components) in a control room in communication with electronics in the magnet room. The MRI Scanner 10S can be any MRI Scanner as is well known to those of skill in the art. The workstation 60 can be in the control room or in the magnet room (shown in the control room in FIG. 3). Controls and display 20 can be in the magnet room for ease of clinician access during a (cardiac) procedure.

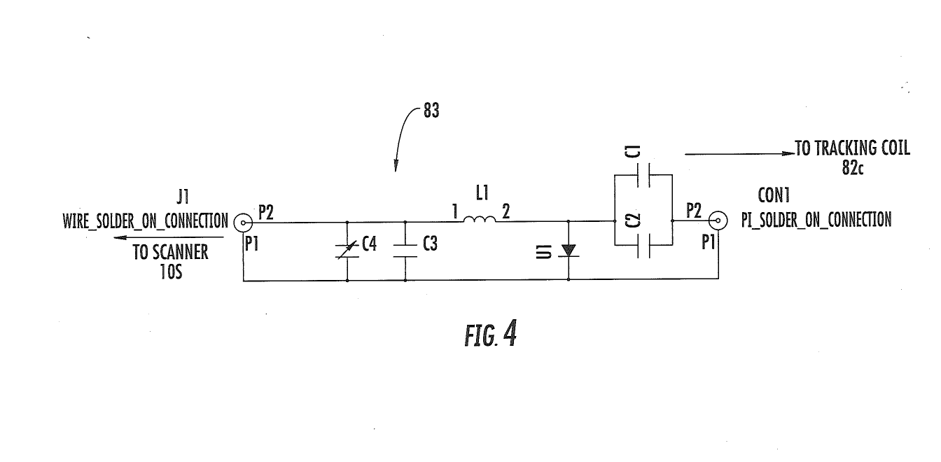

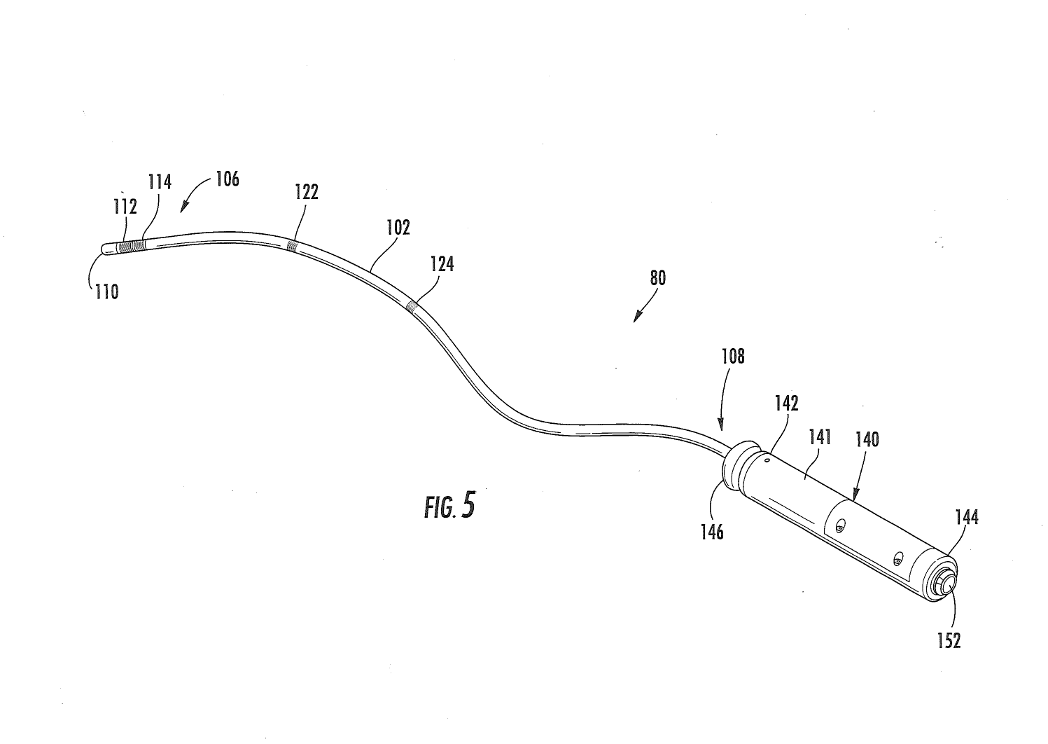

The tracking coils 82c can each include a tuning circuit that can help stabilize the tracking signal for faster system identification of spatial coordinates. FIG. 4 illustrates an example of a tuning circuit 83 that may be particularly suitable for a tracking coil 82c, according to embodiments of the present invention. As shown, CON1 connects the coaxial cable 81 to the tracking coil 82c on a distal end portion of the device 80 while J1 connects to the MR Scanner channel 10ch. The Scanner 10S sends a DC bias to the circuit 83 and turns U1 diode “ON” to create an electrical short which creates a high impedance (open circuit) on the tracking coil 82c to prevent current flow on the tracking coil 82c and/or better tracking signal (stability). The tuning circuit 83 can be configured to have a 50 Ohm matching circuit (narrow band to Scanner frequency) to electrically connect the cable 81 to the respective MR Scanner channel. When the diode U1 is open, the tracking coil data can be transmitted to the MR Scanner receiver channel 10ch. The C1 and C2 capacitors are large DC blocking capacitors. C4 is optional but can allow for fine tuning (typically between about 2-12 picofarads) to account for variability (tolerance) in components. It is contemplated that other tuning circuits and/or tracking signal stabilizer configurations can be used. The tuning circuit 83 can reside in the intrabody device 80 (such as in a handle (e.g., 140, FIG. 5) or other external portion), in a connector that connects the coil 82c to the respective MRI scanner channel 10ch, in the Scanner 10S, in an interface box 86 (FIG. 2), a patch panel 250 (FIG. 3) and/or the tuning circuit 83 can be distributed among two or more of these or other components.

In some embodiments, each tracking coil 82c can be connected to a coaxial cable 81 having a length to the diode via a proximal circuit board (which can hold the tuning circuit and/or a decoupling/matching circuit) sufficient to define a defined odd harmonic/multiple of a quarter wavelength at the operational frequency of the MRI Scanner 10S, e.g., λ/4, 3λ/4, 5λ/4, 7λ/4 at about 123.3 MHz for a 3.0T MRI Scanner. This length may also help stabilize the tracking signal for more precise and speedy localization. The tuned RF coils 82c can provide stable tracking signals for precise localization, typically within about 1 mm or less. Where a plurality (e.g., two closely spaced) of adjacent tracking coils 82c are fixed on a substantially rigid material, the tuned RF tracking coils 82c can provide a substantially constant spatial difference with respect to the corresponding tracking position signals.

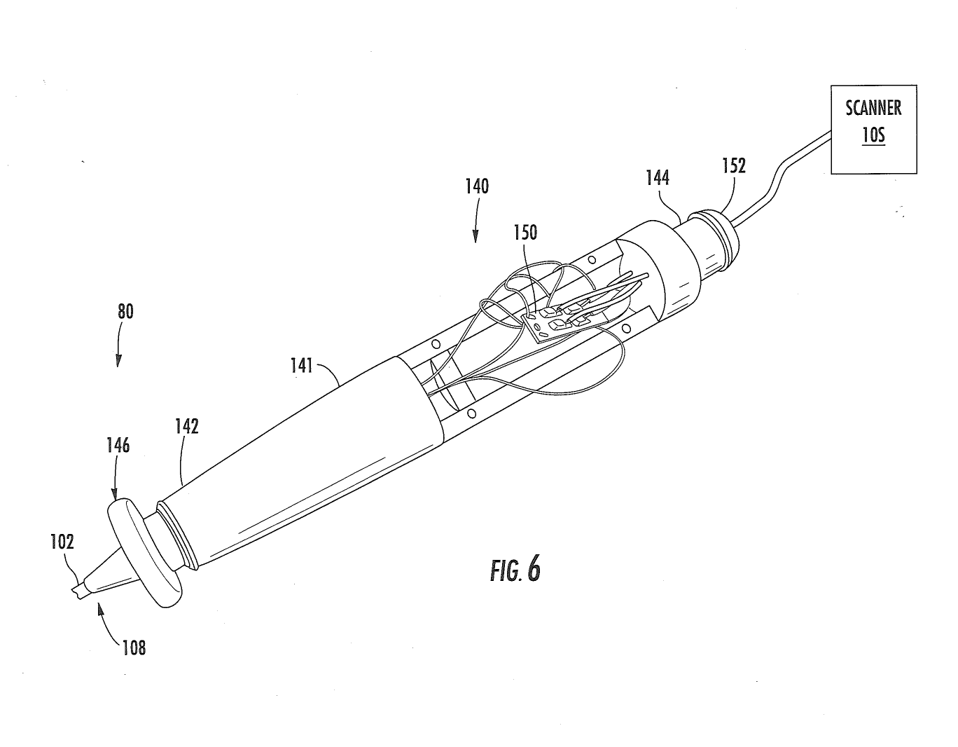

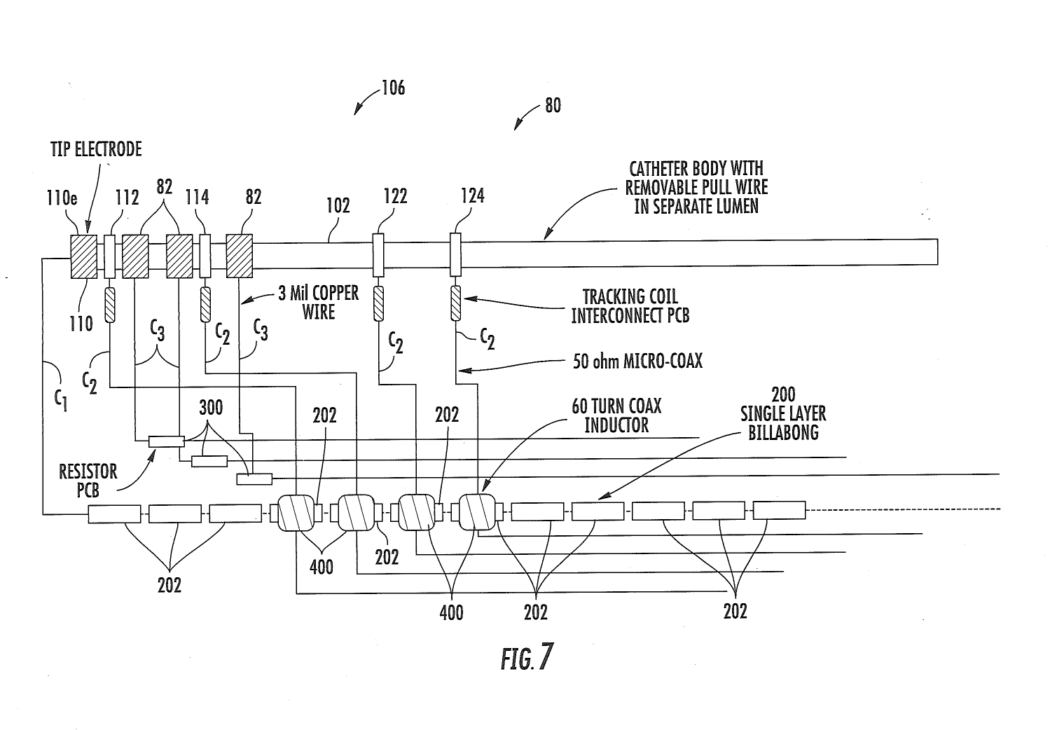

Referring to FIGS. 5-12, a flexible (steerable) catheter 80, such as an ablation catheter, for use in MRI-guided ablation procedures, according to some embodiments of the present invention, is illustrated. The ablation catheter 80 includes an elongated flexible housing or shaft 102 having at least one lumen (not shown) therethrough and includes opposite distal and proximal end portions 106, 108, respectively. The distal end portion 106 includes an ablation tip 110 having an ablation electrode 110e (FIG. 7) for ablating target tissue. A pair of RF tracking coils individually identified as 112, 114, and which are equivalent to coils 82c of FIGS. 2-3, are positioned upstream from the ablation tip 110, as illustrated. The ablation ,tip 110 can include at least one sensing electrode 82 (FIG. 7) for sensing local electrical signals or properties, or an ablation electrode 110e at the ablation tip 110 can be bipolar and both ablate and sense. In some embodiments, the sensing electrodes 82 can be formed from copper or copper alloy (e.g., copper alloy 101, available from McMaster-Carr, Santa Fe Springs, Calif.). In other embodiments, the sensing electrodes 82 can be plated with gold, copper gold, or titanium nitride, for example.

The proximal end portion 108 of the catheter 80 is operably secured to a handle 140 (FIG. 5). The catheter shaft 102 is formed from flexible, bio-compatible and MRI-compatible material, such as, for example, polyester or other polymeric materials. However, various other types of materials may be utilized to form the catheter shaft 102, and embodiments of the present invention are not limited to the use of any particular material. In some embodiments, the shaft proximal end portion 108 is formed from material that is stiffer than the distal end portion 106. The proximal end may be stiffer than a medial portion between the distal and proximal end portions 106, 108. The catheter distal end portion 106 of the ablation catheter 80 can include a second pair of RF tracking coils 122, 124 in spaced apart relationship, as illustrated.

The catheter 80 can be configured to reduce the likelihood of undesired heating caused by deposition of current or voltage in tissue, as will be described below.

The ablation tip electrode 110e is connected to an RF conductor (C₁, FIG. 7) that extends longitudinally within the lumen of the catheter shaft 102 to an electrical connector interface 150 (FIG. 6) within the handle 140 and that connects the ablation electrode 110e to an RF generator. The RF ablation electrode 110e is formed from a conductive material capable of receiving RF energy and ablating tissue. Exemplary materials include copper, copper alloy, as well as bio-compatible materials such as platinum, gold, etc. In some embodiments, the ablation electrode 110e can be copper coated and then plated with gold, copper gold, or titanium nitride, for example. In other embodiments, the ablation tip 110 may include a cryogenic ablation electrode/device configured to cryogenically ablate tissue. For example, the ablation catheter 80 can also or alternatively be configured to apply other ablation energies including cryogenic (e.g., cryoablation), laser, microwave, and even chemical ablation. In some embodiments, the ablation can be carried out using ultrasound energy. In particular embodiments, the ablation may be carried out using HIFU (High Intensity Focused Ultrasound). When MRI is used this is sometimes called Magnetic Resonance-guided Focused Ultrasound, often shortened to MRgFUS. This type of energy using a catheter to direct the energy to the target cardiac tissue can heat the tissue to cause necrosis.

In some embodiments of the present invention, the ablation tip 110 may include one or more exit ports in fluid communication with an irrigation lumen within the catheter shaft 102 and fluid source, for example, at the proximal end portion of the catheter shaft 102, typically at the handle 140. The fluid/solution can provide coolant and/or improve tissue coupling with the ablation tip 110. In some embodiments of the present invention, the ablation tip 110 may be configured to detect temperatures. For example, the ablation tip 110 may include a thermocouple, thermistor, etc.

FIG. 6 is a perspective view of the handle 140, which is connected to the proximal end portion 108 of the catheter shaft 102, according to some embodiments of the present invention. The handle 140 has a main body portion 141 with opposite distal and proximal end portions 142, 144. In FIG. 6, a cover is removed from the handle main body portion 141 to illustrate the termination of the various leads extending into the handle 140 from the shaft lumen at an electrical connector interface 150 (shown as a printed circuit board (PCB)). Electrical connector interface 150 is electrically connected to an adapter 152 at the proximal end 144 of the handle 140. Adapter 152 is configured to receive one or more cables that connect the ablation catheter 80 to an MRI scanner 10S (FIGS. 1-3) and that facilitate operation of the RF tracking coils 112, 114, 122, 124 (FIGS. 5, 7). Adapter 152 also is configured to connect the ablation tip 110 to an ablation source. In the illustrated embodiment, electrical connector interface 150 can also include a decoupling circuit.

In some embodiments of the present invention, RF tracking coils 112, 114, 122, 124 (FIGS. 5, 7) may be between about 2-16 turn solenoid coils. However, other coil configurations may be utilized in accordance with embodiments of the present invention. Each of the RF tracking coils 112, 114, 122, 124 can have the same number of turns or a different number of turns, or different ones of the RF tracking coils 112, 114, 122, 124 can have different numbers of turns. It is believed that an RF tracking coil with between about 2-4 turns at 3.0 T provides a suitable signal for tracking purposes.

Referring now to FIG. 7, the distal end portion 106 of an ablation catheter 80, according to some embodiments of the present invention, is illustrated. The ablation catheter 80 includes an elongated flexible housing or shaft 102 having at least one lumen (not shown) therethrough. The distal end portion 106 includes an ablation tip 110 having an ablation electrode 110e for ablating target tissue. A pair of RF tracking coils individually identified as 112, 114, and which are equivalent to coils 82c of FIGS. 2-3, are positioned upstream from the ablation tip 110, as illustrated. The illustrated catheter distal end portion 106 includes a second pair of RF tracking coils 122, 124 in spaced apart relationship, as illustrated. The illustrated catheter distal end portion 106 includes a pair of EGM (electrogram) sensing electrodes 82 positioned between the first and second tracking coils 112, 114, and a sensing electrode 82 positioned between the tracking coil 114 and the tracking coil 122.

The catheter 80 can include at least the following features for reducing undesired heating caused by RF-induced current: a) a “billabong” cable assembly 200 is used for the RF conductor C₁ to the ablation electrode 110e, and may optionally be used for the electrical conductors (e.g., coaxial cables) C₂ to the tracking coils 112, 114, 122, 124, and the electrical conductors C₃ to the sensing electrodes 82; b) high impedance resistors 300 are used with the sensing electrodes 82; and c) self-resonant cable traps 400 are used with the tracking coil connections.

The billabong cable assembly 200 can include at least the RF conductor C₁ and may also include the various cables/conductors (i.e., C₂, C₃) extending through the lumen of the catheter shaft 102 and connected to the various components of the ablation catheter 80. The billabong cable assembly 200 includes a series of pre-formed back and forth segments 202 in a serpentine shape (e.g., the various conductors C₂, C₃ and RE wire C₁ turn on themselves in a lengthwise direction a number of times along its length). The term “serpentine” refers to a curvilinear shape of pre-formed back and forth turns of a conductor as a subset of a length of the conductor, such as, for example, in an “s” or “z” like shape, including, but not limited to at least one flattened “s” or “z” like shape, including a connected series of “s” or “z” like shapes or with additional sub-portions of same or other curvilinear shapes to define forward and backward sections of a conductor. The upper and lower (and any intermediate) lengthwise extending segments of a serpentine shape may have substantially the same or, different physical lengths.

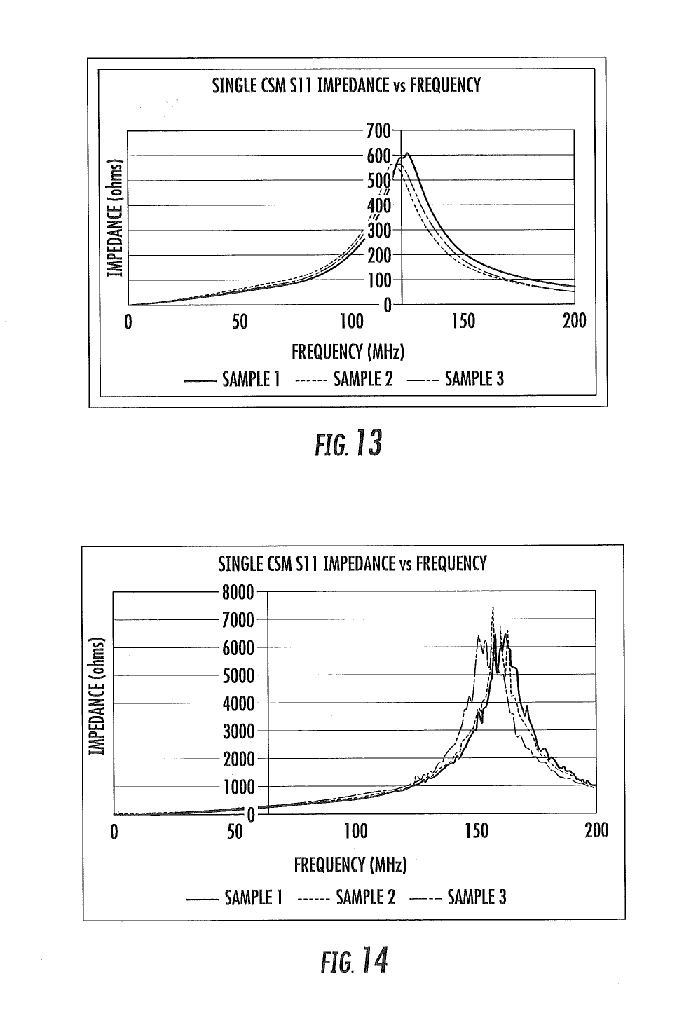

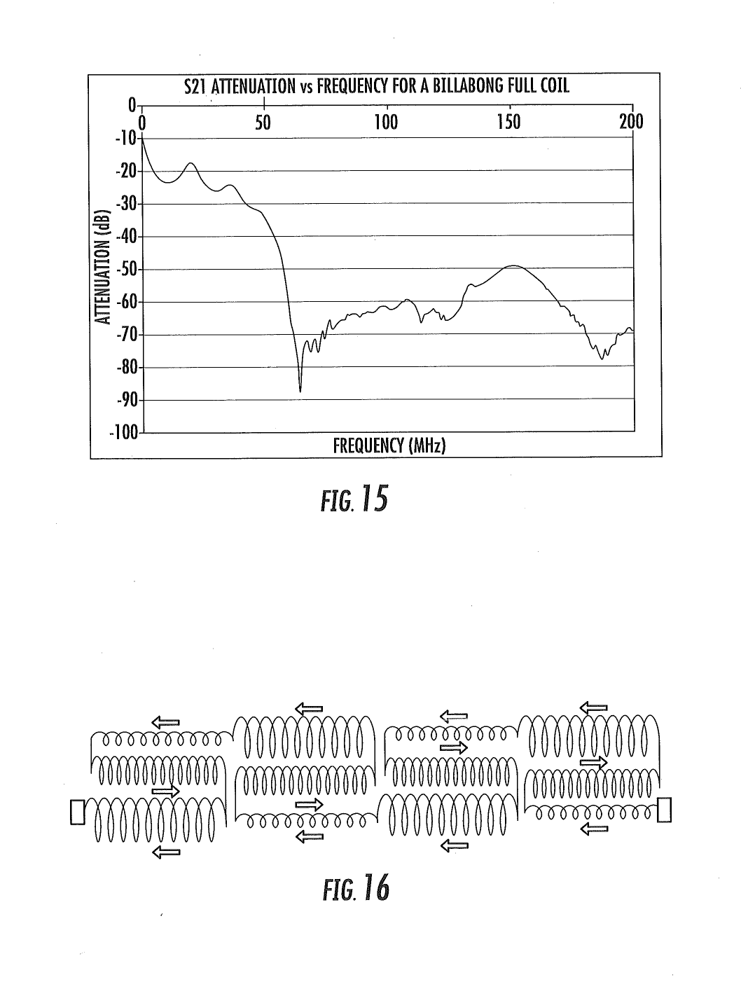

Each of the back and forth segments 202 are referred to as current suppression modules (CSMs). The individual CSMs 202 have frequency responses dependent on length, pitch, and diameter. Responses from different configurations having good RF safety performance are illustrated in FIGS. 13 and 14. A Billabong coil (composed of many CSMs 202) can be configured to deliver broad spectrum high attenuation as illustrated in FIG. 15.

The billabong cable assembly 200 has a unique property of self-cancelling any induced RF current that wants to flow on the cable assembly 200. At the same time, the billabong cable assembly 200 provides a low loss path for the 500 KHz ablation current which can reach about 800 mA.

The billabong cable assembly 200 performs heat management by a combination of mechanisms. For example, each CSM 202 has a high impedance and short length (with respect to the wavelength at MRI frequencies), thus reducing coupling to the local E fields. A CSM's characteristic impedance also provides tank circuit characteristics, as illustrated in FIGS. 13 and 14. The back and forth winding of each CSM 202 results in an increased self inductance of the conductor and a build up of parasitic capacitance between the various winds. This self inductance and stray capacitance cause each CSM 202 to electrically resonate at a particular frequency. Resonating frequency can be chosen to be equal to frequency at which an MRI scanner transmits RF energy. FIG. 13 shows the impedance of a billabong design in which a CSM 202 resonates at approximately 123 MHz, which is the frequency of operation of a 3T MRI scanner. Design of a CSM 202 controls the magnitude of the impedance as well as the resonance frequency. FIG. 14 shows the impedance developed by a CSM 202 that is in the range of 6,000 ohm.

Multiple CSMs 202 in series along the length of the device cancel propagating current by phase cancellation between alternate CSMs 202. Also, multiple CSM billabong conductor/transmission lines have a low pass filter characteristics, such as shown in FIG. 15 (e.g., attenuates RF transmission of frequencies >50 MHz). In addition, the alternating layers of a CSM 202 coiled in opposite directions provide cancellation of common mode current deposited on the CSM conductors. For the billabong design, the coil diameter, pitch and parasitic capacitance resulting from the wound wires affects electrical properties (impedance and peak frequency) for a given CSM length.

In some embodiments, the billabong cable assembly 200 is a single layer billabong assembly, as illustrated in FIG. 16. The illustrated billabong assembly is a conductor having a plurality of closely spaced conductor portions in a serpentine shape.

EGM signals are detected by the sensing electrodes 82 that are in close proximity to cardiac tissue. High impedance (e.g., 5 Kohm or greater) resistors 300 are used to isolate the sensing electrodes 82 from the conductor that connects the electrode assembly to ECG amplifiers. Exemplary resistors 300 are nonmagnetic thick or thin film surface mount types of resistors. ECG amplifiers have very high input impedance (1 MegaOhm), therefore there is negligible signal loss due to 5 Kohm resistors. However, resistors 300 at the sensing electrodes 82 provide significant impedance to any RF induced current that might want to flow through the sensing electrodes 82 to the surrounding tissue.

The tracking coils 112, 114, 122, 124 detect MRI signals in the RF signals. In order to preserve the integrity of a detected MRI signal, the MRI signal is transmitted down the catheter shaft 102 using, for example, 50 ohm coaxial cables. In some embodiments, a tracking coil coaxial cable has a 46AWG, 50 ohm conductive center conductor surrounded by a dielectric layer, and a conductive shield enclosed by an insulating jacket. The coaxial cables C₂ isolate the RF signal transmitted via the coaxial cables C₂ by concentrating the RF signal between the center conductor and the enclosing shield of a respective coaxial cable C₂. The center conductor of a respective coaxial cable C₂ is isolated from outside effects, but the shield of the coaxial cable is susceptible to conducting induced RF currents. As such, according to some embodiments of the present invention, self-resonant cable traps 400 are utilized with the conductors G₂.

Referring to FIG. 8, a self-resonant cable trap 400 is illustrated in more detail. A high impedance point on a coaxial cable shield is created by winding the coaxial cable C₂ as a solenoid such that the inductance of the shield increases to a point where the stray capacitance and the inductance self-resonate at the scanner frequency of operation, which is 128 MHz for a 3T MRI scanner. In some embodiments, each self-resonant cable trap 400 is a 60 turn inductor. The 60 turn inductor has the frequency response of a low pass filter. However, other numbers of turns are possible, typically between about 20-100 turns, according to embodiments of the present invention.

Winding the coaxial cable C₂ as a solenoid (e.g., 60 turns) develops inductance on the shield of the coaxial cable C₂ while the signals traveling inside the coaxial cable C₂ do not see any change. This external inductance prevents RF currents from flowing externally on the shield of the coaxial cable C₂ through the tracking coils (112, 114, 122, 124) thereby reducing local heating around the tracking coils (112, 114, 122, 124).

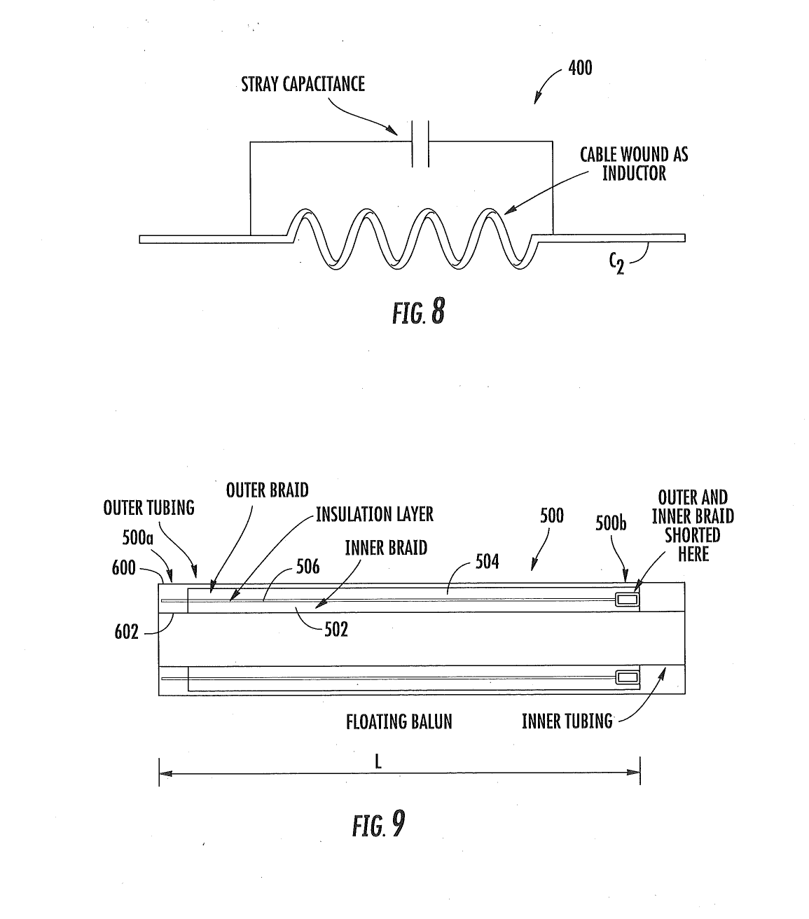

In order to further isolate the conductors (e.g., C₁, C₂, C₃) in an ablation catheter 80 from RF currents induced by the MRI coil, a floating balun or RF shield 500 (FIG. 9) may be embedded within a sheath 600, such as an introducer sheath. An ablation catheter 80 can be then fed through the lumen 602 of the sheath 600, as illustrated in FIG. 10. The illustrated RF shield 500 includes an inner electrical conductor 502 (e.g., a conductive braid) and an outer electrical conductor 504 (e.g., a conductive braid), separated by a dielectric insulator 506. At one end 500b of the RF shield 500, the inner and outer conductors 502, 504 are shorted (i.e., electrically connected). At the other end 500a, the inner and outer conductors 502, 504 are not connected (i.e., the inner and outer conductors 502, 504 are open circuited).

In some embodiments of the present invention, the length L of the RF shield 500 is selected to equal one quarter lambda (¼λ) wavelength of the MRI scanner frequency of operation. Taking into account the effect of electrical insulation on top of the outer conductor 504 and the thickness of the dielectric insulator 506 between the inner and outer conductors 502, 504, the length L is approximately forty eight centimeters (48 cm) for a sheath having an inside diameter of ten French (10F).

Because the inner and outer conductors 502, 504 are shorted at one end 500b and open circuited at the opposite end 500a, induced RF currents encounter high impedance at the shorted end and cannot flow on the outer conductor 504. Moreover, because the outer conductor 504 is electrically conductive, RF currents are prevented from penetrating through to the inner conductor 502 and the central lumen of the sheath 600. As such, the RF shield 500 isolates the portion of conductors (e.g., C₁, C₂, C₃) within an ablation catheter 80 that are surrounded by the RF shield 500.

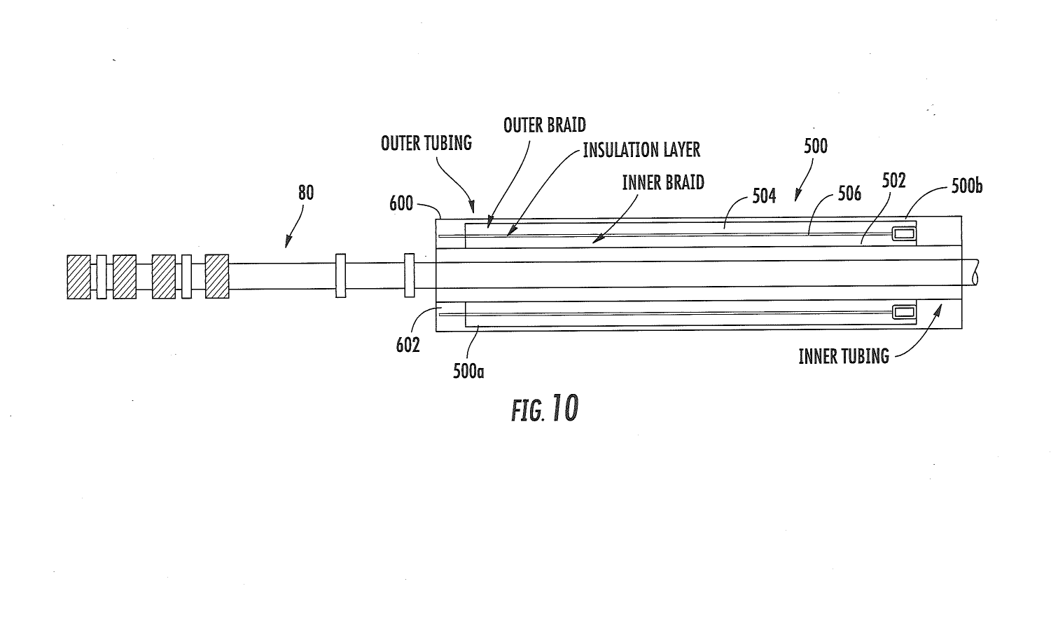

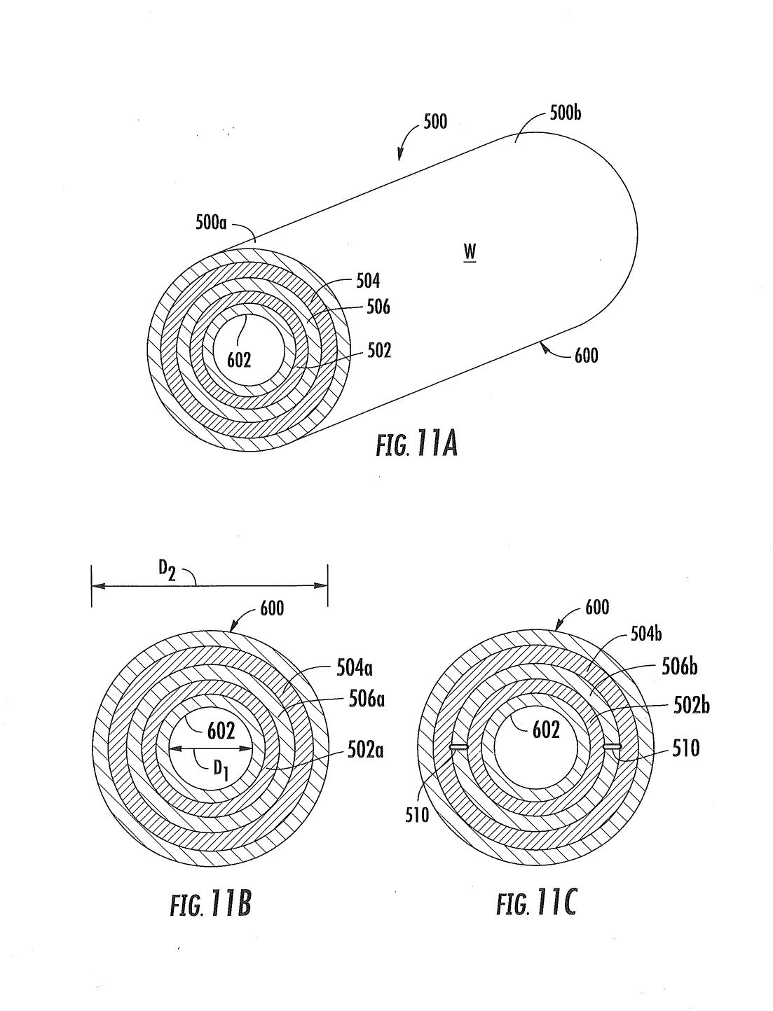

An exemplary RF shield 500, according to some embodiments of the present invention, is illustrated in more detail in FIGS. 11A-11C. The illustrated RF shield 500 is embedded within a wall W of a sheath 600 and has opposite end portions 500a, 500b. FIG. 11A is a perspective view of the RF shield 500, and FIGS. 11B and 11C are respective end views of the RF shield 500. The illustrated RF shield 500 includes an elongated inner tubular conductor 502 having opposite end portions 502a, 502b. An elongated dielectric layer 506 coaxially surrounds the inner tubular conductor 502, and an elongated outer tubular conductor 504 coaxially surrounds the dielectric layer 506 and has opposite end portions 504a, 504b. The inner and outer tubular conductors 502, 504 are electrically connected to each other (i.e., shorted) at only one of the end portions. The opposite respective end portions are electrically isolated from each other. In the illustrated embodiment, the inner and outer tubular conductors 502, 504 are electrically connected to each other at adjacent end portions 502b, 504b. End portions 502a, 504a are electrically isolated from each other.

In some embodiments, the internal diameter D₁ of the sheath 600 may range from between about 0.170 inch and about 0.131 inch; however, other diameters are possible. An outer diameter D₂ of the sheath 600 may range from between about 0.197 inch and about 0.158 inch, and typically between about 5 French and about 12 French (0.066 inch-0.158 inch); however, other diameters are possible. Exemplary thicknesses of the inner and outer conductors 502, 504 may be between about 0.01 inch and about 0.05 inch; however, other thicknesses are possible. Exemplary thicknesses of the dielectric layer 506 may be between about 0.005 inch and about 0.1 inch; however, other thicknesses are possible.

The thickness of the sheath wall W can be relatively thin, such as between about 0.01 inches and about 0.03 inches; however, other thicknesses are possible. The diameter and length of the sheath 600 may vary depending upon the patient and/or the procedure for which the catheter 80 is being utilized. Embodiments of the present invention are not limited to any particular sheath size, length, pr wall thickness of a medical interventional device. The sheath 600 can comprise MRI compatible material, such as flexible polymeric material. Various types of polymeric materials may be utilized and embodiments of the present invention are not limited to the use of any particular type of MRI-compatible material. In some embodiments, the sheath proximal end 500b may be connected to a hemostasis valve (not shown) that is configured to prevent or reduce blood loss and the entry of air, as would be understood by those skilled in the art of the present invention.

The inner and outer tubular conductors 502, 504 may be electrically connected in various ways known to those skilled in the art of the present invention. In the illustrated embodiment, the inner and outer tubular conductors 502, 504 are electrically connected via a pair of jumper wires (or other conductive elements) 510 (FIG. 11C). Jumper wires 510 may be braided wires (e.g., copper wire, copper-plated silver wire, etc.) in some embodiments of the present invention. In other embodiments, the inner and outer tubular conductors 502, 504 may be electrically connected by allowing one of the adjacent end portions 502a, 504a or 502b, 504b to contact each other.

The inner and outer tubular conductors 502, 504 may be formed from various types of non-paramagnetic, conductive material including, but not limited to, conductive foils and conductive braids. In some embodiments, the inner and outer conductors 502, 504 can be formed as thin-film foil layers of conductive material on opposite sides of a thin film insulator (e.g., a laminated, thin flexible body). An exemplary conductive foil is aluminum foil and an exemplary conductive braid is a copper braid. In some embodiments, the inner and outer tubular conductors 502, 504 may be formed from a film having a conductive surface or layer. An exemplary film is Mylar® brand film, available from E. I. DuPont de Nemours and Company Corporation, Wilmington. Del.

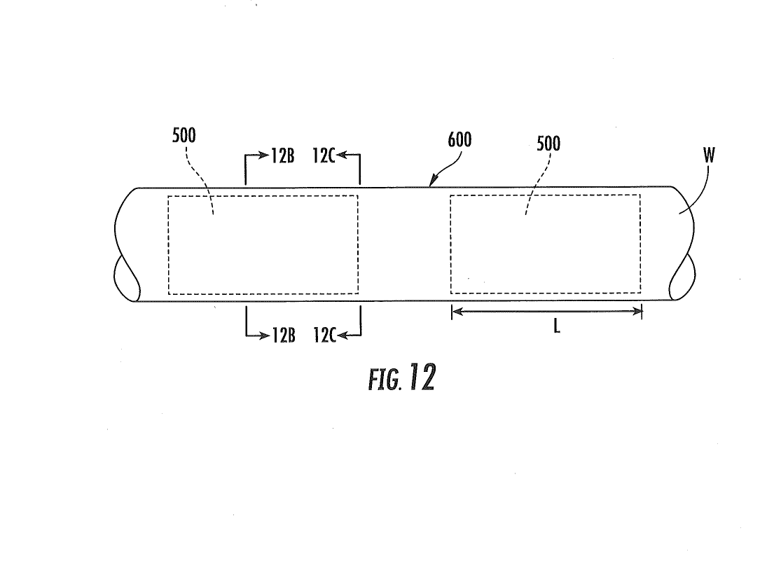

Referring now to FIG. 12, the sheath 600 of FIG. 10 may include a plurality of RF shields 500 coaxially disposed within the wall W thereof in end-to-end spaced-apart relationship. Although a pair of RF shields 500 are illustrated in FIG. 12, it is understood that many additional RF shields 500 may be coaxially disposed within the elongated sheath wall W in end-to-end spaced-apart relationship. Only two RF shields 500 are shown for ease of illustration. The RF shields 500 are spaced-apart sufficiently to allow articulation of the sheath 600 and without any stiff points. In some embodiments, adjacent RF shields 500 may be spaced-apart between about 0.1 inches and about 1.0 inches. For example, adjacent RF shields 10′ may be spaced apart 0.1 inch, 0.15 inch, 0.20 inch, 0.25 inch, 0.30 inch, 0.35 inch, 0.40 inch, 0.45 inch, 0.50 inch, 0.55 inch, 0.60 inch, 0.65 inch, 0.70 inch, 0.75 inch, 0.80 inch, 0.85 inch, 0.90 inch, 0.95 inch, 1.0 inch, etc. Moreover, all adjacent RF shields 10′ may not be spaced apart by the same amount in some embodiments of the present invention. In addition, embodiments of the present invention are not limited to the range of 0.1 inch to 1.0 inch. Other ranges are possible according to some embodiments of the present invention.

In some embodiments of the present invention, one or more RF shields 500, as described above, may be coaxially disposed within the elongated flexible shaft 102 of the catheter 80.

In the drawings and specification, there have been disclosed embodiments of the invention and, although specific terms are employed, they are used in a generic and descriptive sense only and not for purposes of limitation, the scope of the invention being set forth in the following claims. Thus, the foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

1. An MRI-compatible catheter, comprising:
an elongated flexible shaft having a distal end portion, and an opposite proximal end portion;
a handle attached to the proximal end portion, wherein the handle includes an electrical connector interface configured to be in electrical communication with an MRI scanner; and
at least one RF tracking coil positioned adjacent the distal end portion, wherein the at least one tracking coil includes a conductive lead extending between the at least one RF tracking coil and the electrical connector interface and configured to electrically connect the at least one tracking coil to the MRI scanner, and wherein the conductive lead includes a series of pre-formed back and forth segments along its length; and
at least one sensing electrode at the shaft distal end portion.

2. The catheter of claim 1, wherein the conductive lead is a coaxial cable.

3. The catheter of claim 2, wherein the coaxial cable comprises a self-resonant cable trap, and wherein the coaxial cable comprises a self-resonant cable trap.

4-5. (canceled)

6. The catheter of claim 1, further comprising an ablation tip at the shaft distal end portion, and wherein an RF conductor extends longitudinally within the shaft to the electrical connector interface at the handle and that connects the ablation tip to an RF generator.

7. The catheter of claim 6, further comprising a thermocouple at the shaft distal end portion.

8. The catheter of claim 6, further comprising at least one fluid exit port at the shaft distal end portion, wherein the at least one fluid exit port is in fluid communication with an irrigation lumen that extends longitudinally through the catheter shaft lumen from the at least one exit port to the proximal end portion of the catheter shaft, wherein the irrigation lumen is in fluid communication with a fluid/solution source at the proximal end portion of the catheter shaft.

9. The catheter of claim 6, wherein the RF conductor includes a series of pre-formed back and forth segments along its length.

10. (canceled)

11. The catheter of claim 1, wherein the at least one RF tracking coil comprises a tuning circuit configured to stabilize a tracking signal generated by the at least one RF tracking coil.

12. (canceled)

13. The catheter of claim 1, further comprising a sheath surrounding at least a portion of the elongated shaft, wherein the sheath includes at least one RF shield coaxially disposed therewithin, the at least one RF shield comprising:
elongated inner and outer conductors, each having respective opposite first and second end portions, wherein the inner and outer conductors comprise conductive foil, conductive braid, or a film with a conductive surface; and
an elongated dielectric layer of MRI compatible material sandwiched between the inner and outer conductors and surrounding the inner conductor, wherein only the respective first end portions of the inner and outer conductors are electrically connected, and wherein the second end portions are electrically isolated.

14-15. (canceled)

16. The catheter of claim 1, further comprising at least one RF shield coaxially disposed within the flexible shaft, the at least one RF shield comprising:
elongated inner and outer conductors, each having respective opposite first and second end portions, wherein the inner and outer conductors comprise conductive foil, conductive braid, or a film with a conductive surface; and
an elongated dielectric layer of MRI compatible material sandwiched between the inner and outer conductors and surrounding the inner conductor, wherein only the respective first end portions of the inner and outer conductors are electrically connected, and wherein the second end portions are electrically isolated.

17-18. (canceled)

19. An MRI-compatible catheter, comprising:
an elongated flexible shaft having a distal end portion, and an opposite proximal end portion;
a handle attached to the proximal end portion, wherein the handle includes an electrical connector interface configured to be in electrical communication with an MRI scanner;
at least one RF tracking coil positioned adjacent the distal end portion, wherein the at least one tracking coil includes a conductive lead extending between the at least one RF tracking coil and the electrical connector interface and configured to electrically connect the at least one tracking coil to the MRI scanner;
at least one sensing electrode at the shaft distal end portion, wherein the at least one sensing electrode is electrically connected to a resistor having an impedance of at least about 5,000 ohms; and
a sheath surrounding at least a portion of the elongated shaft, wherein the sheath includes at least one RF shield coaxially disposed therewithin, the at least one RF shield comprising:
elongated inner and outer conductors, each having respective opposite first and second end portions; and
an elongated dielectric layer of MRI compatible material sandwiched between the inner and outer conductors and surrounding the inner conductor, wherein only the respective first end portions of the inner and outer conductors are electrically connected, and wherein the second end portions are electrically isolated.

20. The catheter of claim 19, wherein the conductive lead is a coaxial cable that comprises a series of pre-formed back and forth segments along its length.

21. (canceled)

22. The catheter of claim 20, wherein the coaxial cable comprises a self-resonant cable trap, wherein the self-resonant cable trap comprises a 60-turn inductor.

23-24. (canceled)

25. The catheter of claim 19, further comprising an ablation tip at the shaft distal end portion, and wherein an RF conductor extends longitudinally within the shaft to the electrical connector interface at the handle and that connects the ablation tip to an RF generator.

26. The catheter of claim 25, wherein the RF conductor includes a series of pre-formed back and forth segments along its length.

27. The catheter of claim 19, wherein the inner and outer conductors comprise conductive foil, conductive braid, or a film with a conductive surface.

28. (canceled)

29. The catheter of claim 19, wherein the at least one RF tracking coil comprises a tuning circuit configured to stabilize a tracking signal generated by the at least one RF tracking coil.

30. (canceled)

31. An MRI-compatible ablation catheter, comprising:
an elongated flexible shaft having a distal end portion, and an opposite proximal end portion;
a handle attached to the proximal end portion, wherein the handle includes an electrical connector interface configured to be in electrical communication with an MRI scanner and an ablation energy source;
an ablation tip at the shaft distal end portion;
an RF conductor extending longitudinally within the shaft to the electrical connector interface at the handle that connects the ablation tip to an RF generator, wherein the RF conductor includes a series of pre-formed back and forth segments along its length;
at least one RF tracking coil positioned adjacent the distal end portion, wherein the at least one tracking coil includes a conductive lead extending between the at least one RF tracking coil and the electrical connector interface and configured to electrically connect the at least one tracking coil to the MRI scanner, and wherein the conductive lead includes a series of pre-formed back and forth segments along its length; and
a sheath surrounding at least a portion of the elongated shaft, wherein the sheath includes at least one RF shield coaxially disposed therewithin.

32. The ablation catheter of claim 31, wherein the conductive lead is a coaxial cable.

33. The ablation catheter of claim 32, wherein the coaxial cable comprises a self-resonant cable trap, wherein the self-resonant cable trap comprises a 60-turn inductor.

34-35. (canceled)

36. The ablation catheter of claim 31, wherein the at least one RF tracking coil comprises a tuning circuit configured to stabilize a tracking signal generated by the at least one RF tracking coil.

37. (canceled)

38. The ablation catheter of claim 31, wherein the at least one RF shield comprises:
elongated inner and outer conductors, each having respective opposite first and second end portions, wherein the inner and outer conductors comprise conductive foil, conductive braid, or a film with a conductive surface; and
an elongated dielectric layer of MRI compatible material sandwiched between the inner and outer conductors and surrounding the inner conductor, wherein only the respective first end portions of the inner and outer conductors are electrically connected, and wherein the second end portions are electrically isolated.

39-40. (canceled)

41. The ablation catheter of claim 31, further comprising at least one sensing electrode at the shaft distal end portion, wherein the at least one sensing electrode is electrically connected to a resistor having an impedance of at least about 5,000 ohms;
a thermocouple at the shaft distal end portion; and
at least one fluid exit port at the shaft distal end portion, wherein the at least one fluid exit port is in fluid communication with an irrigation lumen that extends longitudinally through the catheter shaft lumen from the at least one exit port to the proximal end portion of the catheter shaft, wherein the irrigation lumen is in fluid communication with a fluid/solution source at the proximal end portion of the catheter shaft.

42-43. (canceled)
